(19) United States
(12) Patent Application Publication
(10) Pub. No.: US 2010/0082053 A1
(43) Pub. Date: Apr. 1, 2010

(54) FLUID EJECTION DEVICE, FLUID EJECTION METHOD AND FLUID EJECTION SURGICAL INSTRUMENT

(75) Inventors: Yasuyoshi HAMA, Shimosuwa-machi (JP); Kinya MATSUZAWA, Shiojiri-shi (JP); Takeshi SETO, Chofu-shi (JP); Hideki KOJIMA, Matsumoto-shi (JP); Yasuhiro ONO, Matsumoto-shi (JP)

Correspondence Address: OLIFF & BERRIDGE, PLC, P.O. BOX 320850, ALEXANDRIA, VA, 22320-4850, US

(73) Assignee: SEIKO EPSON CORPORATION, Tokyo (JP)

(21) Appl. No.: 12/559,046

(22) Filed: Sep. 14, 2009

(30) Foreign Application Priority Data
Sep. 30, 2008 (JP) 2008-252676

(51) Int. Cl. A61B 17/3203

(52) U.S. Cl. 606/167

(57) ABSTRACT

A fluid ejection device includes: a fluid chamber into which fluid flows; a volume varying unit that varies the volume of the fluid chamber; an inlet fluid channel and an outlet fluid channel intercommunicating with the fluid chamber; a fluid ejection port that ejects fluid flowing out from the outlet fluid channel; a fluid supply unit that supplies fluid into the inlet fluid channel; a controller that controls the variation of the volume of the fluid chamber by the volume varying unit; and a color detector that detects color of a fluid ejection target area facing the fluid ejection port, wherein the controller controls the variation of the volume of the fluid chamber by the volume varying unit in accordance with the color detected by the color detecting unit.

Japanese Patent Application No. 2008-252676 filed on Sep. 30, 2008, is hereby incorporated by reference in its entirety.

### BACKGROUND

1. Technical Field

The present invention relates to a fluid ejection device, a fluid ejection method and a fluid ejection surgical instrument in which pressurized fluid is ejected.

2. Related Art

As this type of technique is hitherto known a fluid ejection device including a fluid channel pipe having a connection fluid channel intercommunicating with a fluid ejection port, a pulsation generator having a fluid chamber intercommunicating with the connection fluid channel and a piezoelectric element for changing the volume of the fluid chamber, and a fluid supply unit for supplying fluid into the fluid chamber (see JP-A-2008-082202), for example.

According to this fluid ejection device, the fluid supply unit supplies fluid into the fluid chamber, and the piezoelectric element changes the volume of the fluid chamber, whereby flow of pulsating fluid (hereinafter referred to “pulsation flow”) can be ejected from the fluid ejection port.

Furthermore, as a fluid ejection device for incising or cutting out a biomedical tissue by ejecting fluid is known a fluid ejection device including a nozzle for ejecting fluid and a suction pipe for sucking tissue fragments, etc. broken by ejection of the fluid which are arranged in parallel to or concentrically with each other so that blood clots and tissue fragments busted by ejection of fluid are withdrawn without being scattered to other sites, thereby enhancing the effect and safety of medical treatments (see JP-A-2003-000713).

However, including the techniques disclosed in the above-described publications, a fluid ejection device which can incise or cut out a target site by ejecting fluid has a problem that a site which is not targeted to be incised or cut out (that it, the site which is not prohibited from being incised or cut out) may be erroneously incised or cut out in accordance with the operator's (user's) skill level (for example, when the operator's skill level is low) because incision or cut-out of a target site is performed on the basis of an operator's (user's) judgment (will).

That is, it has been difficult to surely incise or cut out a target site with high precision when the target site is incised or cut out by ejecting fluid to the target site.

### SUMMARY

An advantage of some aspects of the invention is to provide a fluid ejection device, a fluid ejection method and a fluid ejection surgical instrument that can more surely eject fluid with high precision when the fluid is ejected to a target site.

A first aspect of the invention is directed to a fluid ejection device including: a fluid chamber into which fluid flows; a volume varying unit that varies the volume of the fluid chamber; an inlet fluid channel and an outlet fluid channel intercommunicating with the fluid chamber; a fluid ejection port that ejects fluid flowing out from the outlet fluid channel; a fluid supply unit that supplies fluid into the inlet fluid channel; a controller that controls the variation of the volume of the fluid chamber by the volume varying unit; and a color detector that detects color of a fluid ejection target area facing the fluid ejection port, wherein the controller controls the variation of the volume of the fluid chamber by the volume varying unit in accordance with the color detected by the color detecting unit.

As described above, the variation of the volume of the fluid chamber by the volume varying unit is controlled in accordance with the color of the ejection target area. Therefore, a pulsation flow having a characteristic suitable for the property of a fluid ejection target can be ejected to the fluid ejection target site, thereby performing high-precision fluid ejection.

Here, a fluid chamber 501 described later corresponds to the fluid chamber, for example. A piezoelectric element 401 described later corresponds to the volume varying unit, for example. An inlet fluid channel 503 described later corresponds to the inlet fluid channel, for example. An outlet fluid channel 511 described later corresponds to the outlet fluid channel, for example. A fluid ejection opening portion 212 described later corresponds to the fluid ejection port, for example. A pump 20 and a fluid container 10 described later correspond to the fluid supply unit, for example. A controller 30 described later corresponds to the controller, for example. A color sensor 40 described later corresponds to the color detecting unit, for example.

A second aspect of the invention is directed to the fluid ejection device of the first aspect, wherein the controller controls the variation of the volume of the fluid chamber by the volume varying unit so that discharge of fluid generated by the variation of the volume of the fluid chamber is stopped when the color detected by the color detecting unit is a color representing an ejection prohibited area at an ejection target site.

Accordingly, when the ejection target area is an ejection prohibited area, the discharge of fluid generated by the variation of the volume of the fluid chamber can be stopped, and thus an operator can be prevented from erroneously ejecting fluid to the ejection prohibited area. Accordingly, for example when the fluid ejection device is applied to a water pulse surgical knife for surgery, the operator can be prevented from erroneously incising or cutting out an incision/cut-out prohibited area, whereby an ejection target site can be more surely incised or cut out with high precision.

A third aspect of the invention is directed to the fluid ejection device of the first or second aspect, wherein the controller controls the variation of the volume of the fluid chamber by the volume varying unit so that a pulsation flow having a characteristic changeable in accordance with the color detected by the color detecting unit is ejected when the color detected by the color detecting unit is a color representing an ejection permitted area at an ejection target site.

Accordingly, when the ejection target area is an ejection permitted area, the characteristic of the pulsation flow to be ejected, such as an ejection pressure, an ejection period, etc. can be changed in accordance with the color of the ejection target area, and the pulsation flow having the characteristic suitable for the property of a fluid ejection target can be ejected, so that high-precision fluid ejection can be performed.

A fourth aspect of the invention is directed to the fluid ejection device of any of the first to third aspects, which further includes a shape data obtaining unit that obtains data representing the shape of a fluid ejection target site, an area setting unit that sets an ejection permitted area and an ejection prohibited area at an ejection target site on the basis of the data representing the shape of the ejection target site obtained by the shape data obtaining unit, an area determining unit that determines which one of the ejection permitted area and the ejection prohibited area corresponds to the fluid ejection target area, and a discharge suppression unit that suppresses discharge of fluid generated by the variation of the volume of the fluid chamber when the area determining unit determines that the fluid ejection target area corresponds to the ejection prohibited area.

As described above, when it is determined on the basis of the data representing the shape of the ejection target site obtained by the shape data obtaining unit that the ejection target area corresponds to an ejection prohibited area, the discharge of the fluid generated by the variation of the volume of the fluid chamber is suppressed, whereby a fail safe function can be added.

Here, a three-dimensional image creator 801 described later corresponds to the shape data obtaining unit, for example. An area setting unit 802 described later corresponds to the area setting unit, for example. A cut-out state comparator 807 described later corresponds to the area determining unit, for example. The cut-out state comparator 807 described later corresponds to the discharge suppressing unit, for example.

A fifth aspect of the invention is directed to the fluid ejection device of any of the first to fourth aspects, which further includes an informing unit that informs to an operator (user) by using at least one of sound and light, wherein the informing unit informs to the operator (user) when the fluid ejection target area corresponds to any one of an ejection prohibited area and a neighboring area corresponding to an area in the neighborhood of the ejection prohibited area.

Accordingly, it can be informed to the operator that the ejection target area corresponds to an ejection prohibited area or a neighboring area thereof, and the operator can be surely prevented from erroneously ejecting fluid to the ejection prohibited area.

Here, blue LED 100a, red LED 100b and a speaker 100c described later correspond to the informing unit, for example.

A sixth aspect of the invention is directed to a fluid ejection method including: supplying fluid into a fluid chamber; ejecting pressurized fluid from a fluid ejection port by varying the volume of the fluid chamber; detecting color of a fluid ejection target area facing the fluid ejection port; and controlling the variation of the volume of the fluid chamber in accordance with the detected color.

Accordingly, a pulsation flow having a characteristic suitable for the property of a fluid ejection target can be ejected, and thus high-precision fluid ejection can be performed.

A seventh aspect of the invention is directed to a fluid ejection surgical instrument including: a fluid chamber into which fluid flows; a volume varying unit that varies the volume of the fluid chamber; an inlet fluid channel and an outlet fluid channel intercommunicating with the fluid chamber; a fluid ejection port that ejects fluid flowing out from the outlet fluid channel; a fluid supply unit that supplies fluid into the inlet fluid channel; a controller that controls the variation of the volume of the fluid chamber by the volume varying unit; and a color detector that detects color of a surgery target site facing the fluid ejection port, wherein the controller controls the variation of the volume of the fluid chamber by the volume varying unit in accordance with the color detected by the color detecting unit, and incises or cuts out the surgery target site with the fluid which is ejected from the fluid ejection port in accordance with the variation of the volume of the fluid chamber by the volume varying unit.

Accordingly, there can be implemented a surgical instrument that can more surely incise or cut out a target with high precision when the target is incised or cut out by ejecting fluid to the target.

### BRIEF DESCRIPTION OF THE DRAWINGS

The invention will be described with reference to the accompanying drawings, wherein like numbers reference like elements.

FIG. 1 is a diagram showing the construction of a water pulse surgical knife according to a first embodiment of the invention.

FIG. 2 is a cross-sectional view showing a pulsation generator of the water pulse surgical knife shown in FIG. 1.

FIG. 3 is an exploded perspective view of the pulsation generator shown in FIG. 2.

FIG. 4 is a plan view showing an inlet fluid channel of the pulsation generator shown in FIG. 2, and shows an upper case of the pulsation generator when viewed from the contact surface side between the upper case and a lower case.

FIG. 5 is a block diagram showing the construction of a controller.

FIG. 6 is a flowchart showing control processing executed by the controller in the first embodiment.

FIG. 7 is a diagram showing the construction of a water pulse surgical knife according to a second embodiment of the invention.

FIG. 8 is a diagram showing a state that a cut-out permitted area, a cut-out prohibited area and a neighboring area at an affected site.

FIG. 9 is a flowchart showing control processing executed by a controller in the second embodiment.

### DESCRIPTION OF EXEMPLARY EMBODIMENTS

Embodiments according to the present invention will be described hereunder with reference to the accompanying drawings.

A fluid ejection device according to the embodiment of the invention is applicable to drawing using ink or the like, cleaning of a minute object or structure, incision/cut-out of an object, a surgical knife or the like. In the following embodiments, a fluid ejection device according to the invention is applied to a water pulse surgical knife suitable for incision or cut-out of a biomedical tissue of a surgery target site. Accordingly, fluid used in these embodiments is water, normal saline solution, medicinal solution or the like.

### First Embodiment

### Construction

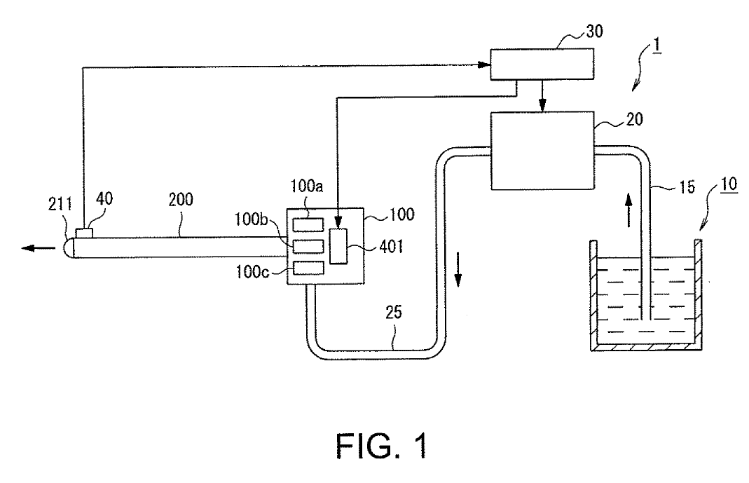

FIG. 1 is a diagram showing the construction of a water pulse surgical knife according to a first embodiment of the invention.

A water pulse surgical knife (fluid ejection device, fluid ejection surgical instrument) 1 shown in FIG. 1 has a fluid container 10 in which fluid is stocked, a pump 20 for supplying fluid under fixed pressure, a pulsation generator 100 for pulsating fluid supplied from the pump 20 and making pulsated fluid flow, a controller 30 for controlling the pulsation generator 100 and the pump 20, and a color sensor 40.

The fluid container 10 stocks fluid such as water, normal saline solution, medicinal solution or the like therein. The pump 20 sucks fluid stocked in the fluid container 10 through a connection tube 15. Furthermore, the pump 20 supplies the sucked fluid through the connection tube 25 to the pulsation generator 100 under fixed pressure. The discharge pressure of the pump 20 is set to about 3 atm (0.3 MPa) or less.

When a surgical operation is executed by the water pulse surgical knife 1, the pulsation generator 100 is a site which is gripped by an operator. Accordingly, it is preferable that the connection tube 25 extending to the pulsation generator 100 is as flexile as possible. Accordingly, it is preferable that the connection tube 25 is a flexible and thin tube and the pressure in the connection tube 25 is set to be low to the extent that fluid can be fed to the pulsation generator 100.

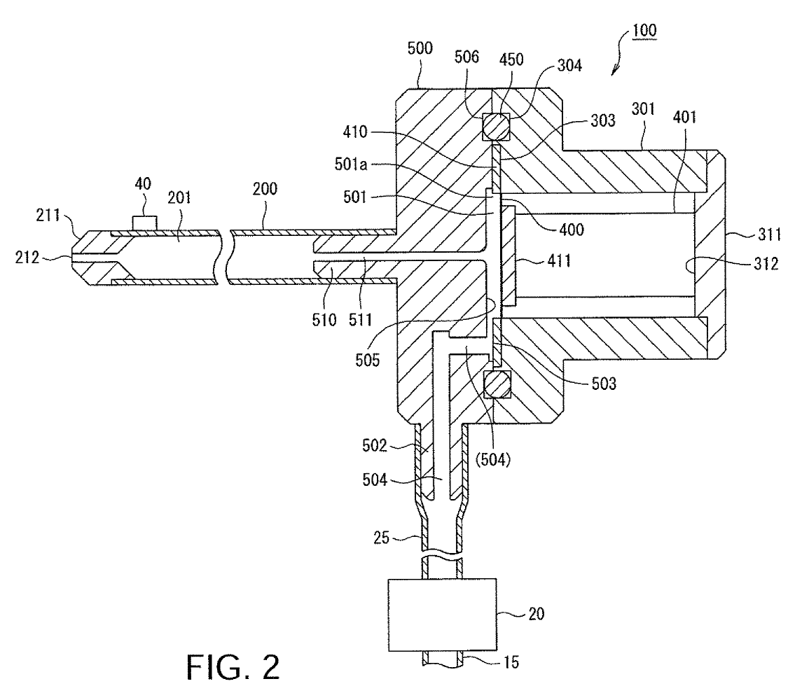

The pulsation generator 100 has a fluid chamber 501 (see FIG. 2), and a volume varying unit for the fluid chamber 501. In this embodiment, a piezoelectric element 401 is used as the volume varying unit of the fluid chamber 501. Furthermore, the pulsation generator 100 contains blue and red LEDs (Light Emitting Diode) 100a, 100b for informing whether it is possible (proper) to incise or cut-out a target site, and a speaker 100c for outputting sounds.

Next, the pulsation generator 100 will be described in more detail with reference to the drawings.

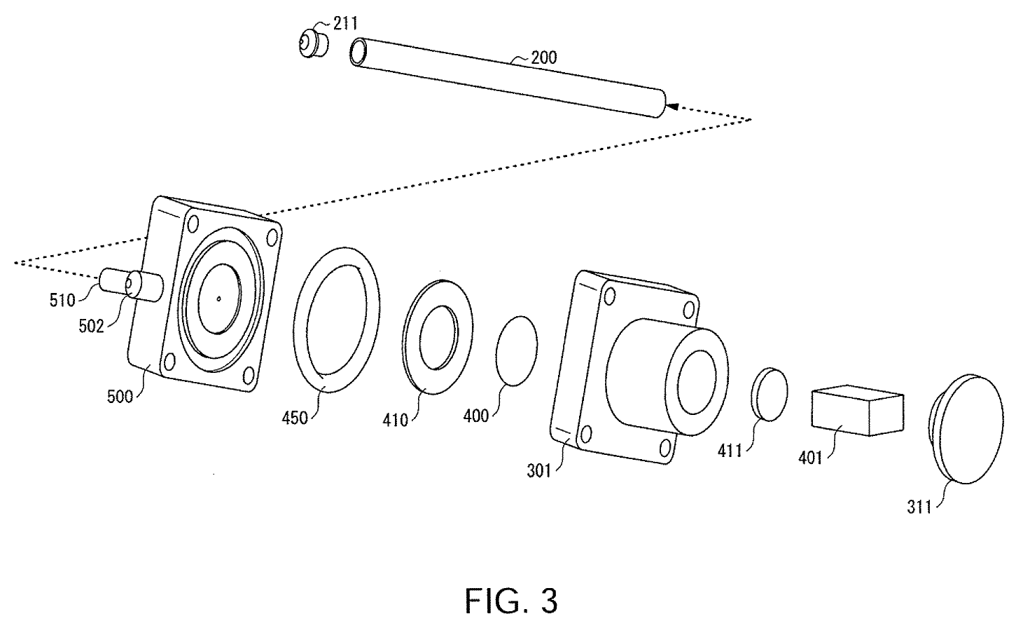

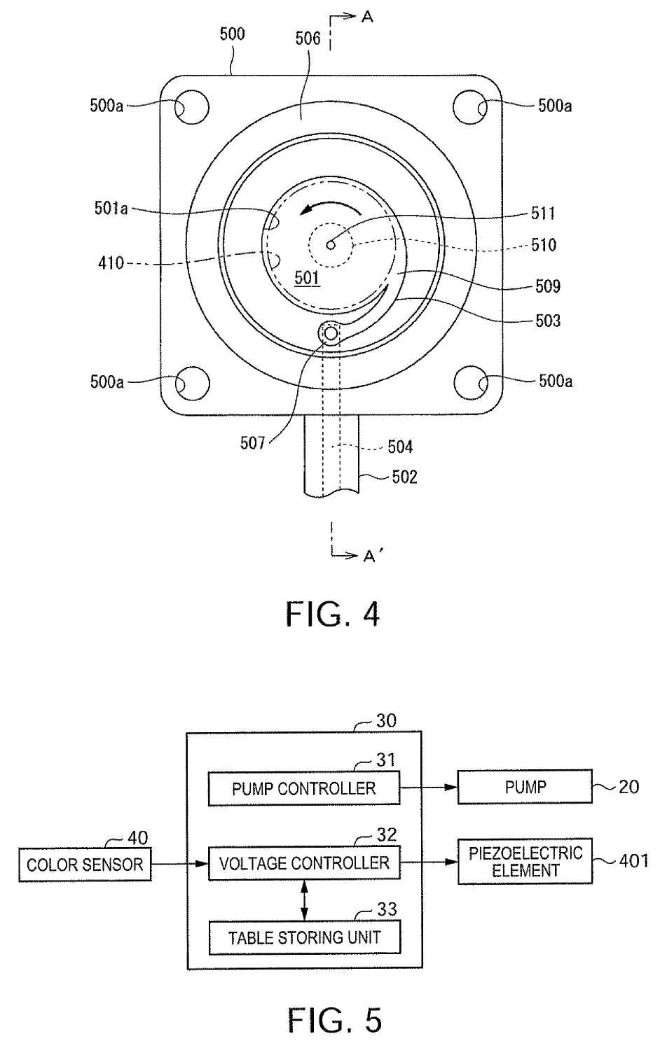

FIG. 2 is a cross-sectional view showing the pulsation generator of the water pulse surgical knife shown in FIG. 1. FIG. 2 is a cross-sectional view taken along A-A′ line of FIG. 4, and FIG. 3 is a perspective view showing a state that the pulsation generator shown in FIG. 2 is exploded.

As shown in FIGS. 2 and 3, the pulsation generator 100 has an upper case 500 and a lower case 301. The upper case 500 and the lower case 301 are joined to each other through confronting faces thereof, and threadably fixed to each other by four fixing screws (not shown).

The lower case 301 is a cylindrical member having a flange portion, and one end portion thereof is hermetically sealed by a bottom plate 311. The piezoelectric element 401 is disposed in the internal space of the lower case 301.

The piezoelectric element 401 is a laminate type piezoelectric element, and constitutes an actuator. One end portion of the piezoelectric element 401 is fixed to a diaphragm 400 through an upper plate 411. The other end portion of the piezoelectric element 401 is fixed to the upper surface 312 of the bottom plate 311.

The diaphragm 400 comprises a disc-shaped metal thin plate. In a recess portion 303 of the lower case 301, the peripheral edge portion of the diaphragm 400 is fixed to the bottom surface of the recess portion 303 in close contact with the bottom surface. A driving signal is input to the piezoelectric element 401 as the volume varying unit, whereby the volume of the fluid chamber 501 is varied through the diaphragm 400 in connection with expansion and contraction of the piezoelectric element 401. As described above, the pulsation generator 100 is designed so that the piezoelectric element 401 and the diaphragm 400 are adopted as the volume varying unit, so that the structure of the pulsation generator 100 can be simplified and thus the pulsation generator 100 can be miniaturized. Furthermore, the maximum frequency of the volume variation of the fluid chamber 501 can be set to a high frequency which is equal to 1 KHz or more, and thus the pulsation generator of this embodiment is optimum to ejection of high-speed pulsation flow.

An reinforcing plate 410 comprising a disc-shaped metal thin plate having an opening portion at the center portion thereof is laminated on the upper surface of the diaphragm 400.

The upper case 500 has a recess portion at the center portion of a surface thereof which faces the lower case 301. A rotational-body type space which is defined by this recess portion and the diaphragm 400 and filled with fluid serves as the fluid chamber 501. That is, the fluid chamber 501 is the space surrounded by a sealing surface 505 of the recess portion of the upper case 500, an inner peripheral side wall 501a and the diaphragm 400. An outlet fluid channel 511 is formed substantially at the center portion of the fluid chamber 501.

The outlet fluid channel 511 penetrates from the fluid chamber 501 till the end portion of an outlet fluid channel pipe 510 projected from one end face of the upper case 500. The connection portion of the outlet fluid channel 511 to the sealing surface 505 of the fluid chamber 501 is smoothly rounded to reduce fluid resistance.

In this embodiment (see FIG. 2), the fluid chamber 501 is designed to have a substantially cylindrical shape sealed at both the ends thereof. However, it may be designed to be conical, trapezoidal, semispherical or the like in side view. For example, when the connection portion between the outlet fluid channel 511 and the sealing surface 505 is designed to be funnel-shaped, bubbles in the fluid chamber 501 described later are easily discharged.

A connection fluid channel pipe 200 is connected to the outlet fluid channel pipe 510. A connection fluid channel 201 is formed in the connection fluid channel pipe 200. The diameter of the connection fluid channel 201 is set to be larger than the diameter of the outlet fluid channel 511. The thickness of the pipe portion of the connection fluid channel pipe 200 is set so that the connection fluid channel pipe 200 is rigid to the extent that it does not absorb pressure pulsation of liquid.

A nozzle 211 is inserted in the tip portion of the connection fluid channel pipe 200, and a fluid ejection port 212 is formed in the nozzle 211. The diameter of the fluid ejection port 212 is set to be smaller than the diameter of the connection fluid channel 201.

Furthermore, the color sensor 40 is disposed in the neighborhood of the nozzle 211. For example, a combination of a photodiode and color filters (RGB) is used as the color sensor 40, and a photodetecting portion is disposed to face in the fluid ejection direction, whereby the color of a cut-out target area (fluid ejection target area) at a surgery target site (ejection target site) can be detected. Here, the cut-out target area is an area which faces the fluid ejection port 212 at the surgery target site and is to be cut out when fluid is ejected.

An inlet fluid channel pipe 502 in which a connection tube 25 for supplying fluid from the pump 20 is inserted is formed in the side surface of the upper case 500 so as to project from the side surface. A connection fluid channel 504 at the inlet fluid channel side is formed in the inlet fluid channel pipe 502. The connection fluid channel 504 intercommunicates with the inlet fluid channel 503. The inlet fluid channel 503 is formed like a groove at the peripheral edge portion of the sealing surface 505 of the fluid chamber 501, and intercommunicates with the fluid chamber 501.

At the joint face between the upper case 500 and the lower case 301, a packing box 304 is formed at the lower case 301 side and a packing box 506 is formed at the upper case 500 side so as to be far away from the diaphragm 400 in the outer peripheral direction. A ring-shaped packing 450 is mounted in a space defined by the packing box 304 and the packing box 506.

Here, when the upper case 500 and the lower case 301 are assembled with each other, the peripheral edge portion of the diaphragm 400 and the peripheral edge portion of the reinforcing plate 410 are brought into close contact with each other by the peripheral edge portion of the sealing surface 505 of the upper case 500 and the bottom surface of the recess portion 303 of the lower case 301. At this time, the packing 450 is pressed under pressure by the upper case 500 and the lower case 301, thereby preventing fluid leakage from the fluid chamber 501.

When fluid is discharged, the inside of the fluid chamber 501 is set to a high pressure state of 30 atm (3 MPa) or more, and thus it may be considered that fluid slightly leaks at the respective joint portions of the diaphragm 400, the reinforcing plate 410, the upper case 500 and the lower case 301. However, the fluid leakage at these joint portions is prevented by the packing 450.

When the packing 450 is disposed as shown in FIG. 2, the packing 450 is compressed by the pressure of fluid leaking from the fluid chamber 501 under high pressure, and more strongly pressed against the inner walls of the packing boxes 304 and 506, whereby the leakage of the fluid can be further surely prevented. Accordingly, high pressure increase in the fluid chamber 501 can be kept when the pulsation generator 100 is driven.

Next, the inlet fluid channel 503 formed in the upper case 500 will be described in more detail with reference to the drawings.

FIG. 4 is a plan view showing the inlet fluid channel of the pulsation generator shown in FIG. 2, and shows the upper case when viewed from the contact surface side to the lower case.

As shown in FIG. 4, the inlet fluid channel 503 intercommunicates with the fluid chamber 501 at one end portion thereof, and intercommunicates with the connection fluid channel 504 at the other end portion thereof. A fluid pool 507 is formed at the connection portion between the inlet fluid channel 503 and the connection fluid channel 504. By providing the fluid pool 507 at the connection portion between the inlet fluid channel 503 and the connection fluid channel 504, the effect of the inertance of the connection fluid channel 504 on the inlet fluid channel 503 can be suppressed. The connection portion between the fluid pool 507 and the inlet fluid channel 503 are smoothly rounded to reduce the fluid resistance.

Furthermore, the inlet fluid channel 503 intercommunicates with the fluid chamber 501 so as to extend substantially tangentially with respect to the inner peripheral side wall 501a of the fluid chamber 501. Fluid supplied from the pump 20 (see FIG. 1) under fixed pressure flows along the inner peripheral side wall 501a (the direction indicated by an arrow in FIG. 4), and generates a swirling flow in the fluid chamber 501. The swirling flow is pressed against the inner peripheral side wall 501a by centrifugal force based on swirling, and also bubbles contained in the fluid chamber 501 concentrate on the center portion of the swirling flow.

The bubbles collected at the center portion are excluded from the outlet fluid channel 511. Accordingly, it is preferable that the outlet fluid channel 511 is provided in the neighborhood of the center of the swirling flow, that is, at the center portion of the rotational body shape. In FIG. 4, the inlet fluid channel 503 is designed to be curved in plan view. The inlet fluid channel 503 may intercommunicate with the fluid channel 501 linearly. However, in order to obtain a desired inertance in a narrow space, the fluid channel length of the inlet fluid channel is required to be increased, and thus the inlet fluid channel 503 is curved.

As shown in FIG. 2, the reinforcing plate 410 is disposed between the diaphragm 400 and the peripheral edge portion of the sealing surface 505 at which the inlet fluid channel 503 is formed. The reinforcing plate 410 is provided to enhance the durability of the diaphragm 400. A connection opening portion 509 having a cut-out shape formed at the connection portion of the inlet fluid channel 503 to the fluid chamber 501. Accordingly, when the diaphragm 400 is driven at a high frequency, stress concentrates in the neighborhood of the connection opening portion 509 and thus fatigue destruction may occur. Therefore, the stress concentration on the diaphragm 400 is prevented from occurring by disposing the reinforcing plate 410 having a continuous opening portion having no cut-out portion,

Screw holes 500a are formed at four places of the outer peripheral corner portions of the upper case 500, and the upper case 500 and the lower case 301 are threadably joined to each other at these screw hole positions.

As not shown, the reinforcing plate 410 and the diaphragm 400 may be joined to each other and integrally fixed as a laminate body. When the reinforcing plate 410 and the diaphragm 400 are laminated and integrally fixed to each other, the assembling performance of the pulsation generator 100 can be enhanced, and also a reinforcing effect of the outer peripheral edge portion of the diaphragm 400 can be obtained. Adhesive fixing using adhesive agent, solid-layer diffusion joint, welding, etc. may be adopted as a fixing method. It is more preferable that the reinforcing plate 410 and the diaphragm 400 are brought into close contact with each other at the joint surface therebetween.

FIG. 5 is a block diagram showing the construction of the color sensor 40 and the controller 30.

As shown in FIG. 5, the controller 30 has a pump controller 31 for controlling the pump 20, a voltage controller 32 for controlling the piezoelectric element 401 of the pulsation generator 100, and a table storing unit 33 for storing a lookup table to which the voltage controller 32 refers. A detection signal of the color sensor 40 is input to the voltage controller 32. The pump controller 31 controls the pressure of fluid supplied to the pulsation generator 100 by the pump 20.

The voltage controller 32 controls variation of the volume of the fluid chamber 501 by the piezoelectric element 401. Specifically, the voltage controller 32 refers to the lookup table stored in the table storing unit 33 on the basis of the detection signal of the color sensor 40, and controls the voltage applied to the piezoelectric element 401. Accordingly, the voltage controller 32 can vary the characteristic of the pulsation flow to be ejected in accordance with the color of the cut-out target area. Here, the pulsation flows different in characteristic are pulsation flows which are different in at least one of ejection pressure and ejection period.

In this embodiment, an area (cut-out permitted area) in which a tumor or the like exists at an affected site is colored in advance so that this area is discriminated from areas (cut-out prohibited areas) other than the cut-out permitted area, whereby the characteristic of the pulsation flow to be ejected is changed in accordance with the state or type of the affected site.

A nanocapsule (nanorobot) of nanometer size having a function of sensing the characteristic of a pathologic lesion or pathologic surface (lesion selecting function) is used as a method of coloring the cut-out permitted area. The nanocapsule can transmit any medical agent, gene, protein or the like encapsulated therein to any cell or tissue in a living body. In this embodiment, nanocapsules contaminated with coloring material are ejected intravenously to color only the cut-out permitted area.

At this time, in order to visually clarify the cut-out permitted area, the color is set to be different from that of the cut-out prohibited area. A different color may be set in accordance with the type of the cut-out permitted area. The table storing unit 33 stores the lookup table to which the voltage controller 32 refers. The lookup table stores voltage pulse waveforms of driving signals. Specifically, a voltage value corresponding to one voltage pulse waveform is stored as a digital value in association with each color of cut-out target areas in the lookup table.

Here, a method of designing a lookup table will be described.

When a lookup table is designed, voltage signals to be stored in the lookup table are set on the basis of examples of operative procedures, examples of experiments, etc. so that the characteristic of pulsation flow to be ejected is proper to a predetermined property (state, type) of a surgery target site. Specifically, an approximation calculation between the characteristic of a pre-assumed pulsation flow and the characteristic of pulsation flow which is obtained from the examples of the operative procedures, estimated to be proper and actually ejected to a surgery target site having the predetermined property, and the characteristic of the pre-assumed pulsation flow is updated on the basis of any update reference. When the difference between the characteristic of the pulsation flow obtained as an update result and the characteristic of the pulsation flow obtained from the examples of the operative procedures is within a predetermined range, the characteristic of the pulsation flow obtained as the update result is set as the characteristic of the pulsation flow proper to the predetermined property of the surgery target site.

The voltage signal associated with the characteristic of the pulsation flow thus set is stored in the lookup table while associated with the color corresponding to the property of the surgery target site, and the lookup table thus obtained is stored in the table storing unit 33.

Next, the processing executed by the controller 30 will be described.

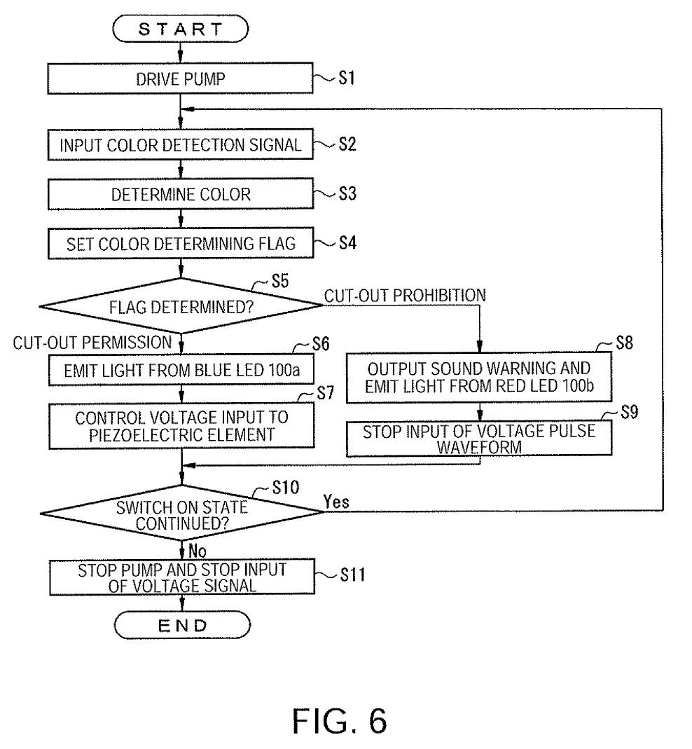

FIG. 6 is a flowchart showing the control processing executed by the controller 30.

Here, the controller 30 repetitively executes the processing shown in FIG. 6 when an operator sets a switch (not shown) for starting ejection of fluid to ON state.

When the switch for starting the fluid ejection is set to ON state by the operator, the pump controller 31 of the controller 30 first drives the pump 20 to supply fluid to the pulsation generator 100 under fixed pressure in step S5 as shown in FIG. 6.

Subsequently, in step S2, the voltage controller 32 of the controller 30 reads in the detection signal of the color sensor 40, and shifts the processing to step S3.

In step S3, on the basis of the detection signal read in step S2, the voltage controller 32 of the controller 30 determines the color of the cut-out target area to which fluid should be ejected. In step S4, the controller 30 sets a color determining flag on the basis of the color determined in step S3. Specifically, when the color determined in step S3 is a color indicated in advance by the operator, it is determined that the cut-out target area corresponds to a cut-out permitted area at a surgery target site, and a cut-out permitting flag is set as the color determining flag. On the other hand, when the color determined in step S3 is a color other than the indicated color, it is determined that the cut-out target area corresponds to a cut-out prohibited area at the surgery target site, and a cut-out prohibiting flag is set as the color determining flag.

Subsequently, in step S5, the controller 30 determines whether the cut-out permitting flag and the cut-out prohibiting flag are set or not, and when the cut-out permitting flag is set, the controller 30 shifts the processing to step S6 to turn on the blue LED 100a and emit light.

Subsequently, in step S7, the controller 30 refers to the lookup table stored in the table storing unit 33 on the basis of the color determined in step S3, determines a voltage signal (driving signal) to be applied to the piezoelectric element 401, inputs the voltage signal concerned to the piezoelectric element 401 and then shifts the processing to step S10 described later.

Accordingly, when the cut-out target area corresponds to the cut-out permitted area, the voltage pulse waveform is input to the piezoelectric element 401 so as to obtain the ejection pressure and the ejection period corresponding to the color of the cut-out target area. As described above, the driving signal is input from the voltage controller 32 to the piezoelectric element 401, whereby pulsed fluid discharge, that is, pulsation flow occurs in the connection fluid channel 201.

Furthermore, in step S5, when the controller 30 determines that the cut-out prohibiting flag is set, the controller 30 shifts the processing to step S8 in which a sound alarm indicating that the cut-out is prohibited is output from the speaker 100c and also light is emitted from the red LED 100b.

Subsequently, in step S9, the controller 30 stops the input of the voltage pulse waveform to the piezoelectric element 401, and shifts the processing to step S10.

In step S10, the voltage controller 32 of the controller 30 determines whether the ON state of the switch for starting the fluid ejection is continued or not. When the ON state is determined to be continued, the processing is shifted to step S2, and when it is determined that the ON state is changed to OFF state, the processing is shifted to step S11.

In step S11, the pump controller 31 of the controller 30 stops the driving of the pump 20, and the voltage controller 32 of the controller 30 stops the input of the voltage signal to the piezoelectric element 401 of the pulsation generator 100 and finishes the series of processing.

Next, the operation of the water pulse surgical knife 1 according to this embodiment will be described.

The discharge of fluid in the pulsation generator 100 of the water pulse surgical knife 1 according to this embodiment is performed on the basis of the difference between the inertance L1 at the inlet fluid channel side (also called as composite inertance L1) and the inertance L2 at the outlet fluid channel side (also called as composite inertance L2).

First, the inertance will be described.

The inertance L is represented by L=ρ×h/S, wherein ρ represents the density of fluid, S represents the cross-sectional area of the fluid channel and h represents the length of the fluid channel. When the pressure difference of the fluid channel is represented by ΔP and the flow amount of the fluid flowing in the fluid channel is represented by Q, the relational expression: ΔP=L×dQ/dt is derived by transforming the dynamic equation in the fluid channel by using the inertance L.

That is, the inertance L represents the degree of an effect applied to the time-variation of the flow amount. The time-variation of the flow amount is smaller as the inertance L is larger, and the time-variation of the flow amount is larger as the inertance L is smaller.

Furthermore, the composite inertance concerning the parallel connection of plural fluid channels and the serial connection of plural fluid channels different in shape can be calculated by combining the inertances of individual fluid channels as in the case of the parallel connection or serial connection of inductances in an electrical circuit.

The connection fluid channel 504 is designed to be sufficiently larger in diameter than the inlet fluid channel 503, and thus the inertance L1 at the inlet fluid channel side is calculated in the range of the inlet fluid channel 503. At this time, the connection tube for connecting the pump 20 and the inlet fluid channel has flexibility, and thus it may be omitted from the calculation of the inertance L1.

The diameter of the connection fluid channel 201 is remarkably larger than that of the outlet fluid channel, and the thickness of the pipe portion (pipe wall) of the connection fluid channel pipe 200 is small, so that the effect on the inertance L2 at the outlet fluid channel side is minor. Accordingly, the inertance L2 at the outlet fluid channel side may be replaced with the inertance of the outlet fluid channel 511. The thickness of the pipe wall of the connection fluid channel pipe 200 is set to provide sufficient rigidity to the pressure propagation of fluid.

In this embodiment, the fluid channel length and cross-sectional area of the inlet fluid channel 503 and the fluid channel length and cross-sectional area of the outlet fluid channel 511 are set so that the inertance L1 at the inlet fluid channel side is larger than the inertance L2 at the outlet fluid channel side.

Next, the operation of the pulsation generator 100 will be described.

When the switch is set to ON state by the operator and the controller 30 starts to drive the pump 20, fluid is supplied to the inlet fluid channel 503 under liquid pressure of fixed pressure at all times by the pump 20. As a result, when the piezoelectric element 401 does not operate, the fluid flows into the fluid chamber 501 due to the difference between the discharge pressure of the pump 20 and the whole fluid resistance value of the fluid at the inlet fluid channel side.

When the controller 30 inputs the driving signal to the piezoelectric element 401, the volume of the fluid chamber 501 is varied through the diaphragm 400 in connection with expansion/contraction of the piezoelectric element 401. That is, when the driving signal is input to the piezoelectric element 401 and the piezoelectric element 401 expands drastically, the pressure in the fluid chamber 501 rapidly increases and reaches several tens atm if the inertances L1 and L2 at the inlet fluid channel side and the outlet fluid channel side are sufficiently large.

This pressure is further larger than the pressure based on the pump 20 which is applied to the inlet fluid channel 503, and thus the flow-in of the fluid from the inlet fluid channel side into the fluid chamber 501 is reduced by the above pressure, so that the flow-out from the outlet fluid channel 511 is increased. Accordingly, the check valve at the inlet fluid channel side which is provided in such a water pulse surgical knife as disclosed in JP-A-2008-082202 may be omitted.

Here, the inertance L1 of the inlet fluid channel 503 is larger than the inertance L2 of the outlet fluid channel 511. Accordingly, the increase amount of the fluid discharged from the outlet fluid channel is larger than the reduction amount of the flow amount of the fluid flowing from the inlet fluid channel 503 into the fluid chamber 501, and thus pulsed fluid discharge, that is, pulsation flow occurs in the connection fluid channel 201. The pressure variation at this discharge time propagates in the connection fluid channel pipe 200, and the fluid is ejected from the fluid ejection port 212 of the nozzle 211 at the tip.

That is, the pulsation generator 100 drives the piezoelectric element 401 to generate pulsation, whereby the fluid supplied from the pump 20 is ejected at high speed through the connection fluid channel pipe 200 and the nozzle 211.

Here, the diameter of the fluid ejection port 212 of the nozzle 211 is smaller than the diameter of the outlet fluid channel 511, and thus the fluid is ejected as pulsed liquid droplets (pulsation flow) at higher pressure and higher speed.

The inside of the fluid chamber 501 is set to a vacuum state immediately after the pressure increases because of the interaction between the reduction of the flow-in amount of the fluid from the inlet fluid channel 503 and the increase of the flow-out of the fluid from the outlet fluid channel 511. As a result, the flow of the fluid in the inlet fluid channel 503 which directs into the fluid chamber 501 at a speed equal to that before the piezoelectric element 401 is operated is restored after lapse of a fixed time by both the pressure of the pump 20 and the vacuum state in the fluid chamber 501.

After the fluid of the fluid flow in the inlet fluid channel 503 is restored, the pulsation flow from the nozzle 211 can be continually ejected when the piezoelectric element 401 expands.

Furthermore, in the operation of the pulsation generator 100 described above, the fluid chamber 501 has the substantially rotational body shape and the inlet fluid channel 503 as the swirling flow generator, and also the outlet fluid channel 511 is opened in the neighborhood of the rotational axis of the substantially rotational body shape, so that the swirling flow occurs in the fluid chamber 501 and bubbles contained in the fluid are quickly discharged from the outlet fluid channel 511 to the outside. Accordingly, even when the volume of the fluid chamber 501 is minutely varied by the piezoelectric element 401, the pressure variation is not disturbed by the bubbles, and sufficient pressure increase is obtained.

Here, in the water pulse surgical knife 1, the color of the cut-out target area is detected, and the voltage signal applied to the piezoelectric element 401 of the pulsation generator 100 is set in accordance with the detected color of the cut-out target area.

In the water pulse surgical knife 1, the voltage controller 32 of the controller 30 controls the piezoelectric element 401 of the pulsation generator 100 on the basis of the voltage signal which is set in accordance with the detected color of the cut-out target area, thereby controlling the voltage applied to the piezoelectric element 401 of the pulsation generator 100 in accordance with the color of the cut-out target area.

As described above, in the water pulse surgical knife 1, the variation of the volume of the fluid chamber 501 based on the piezoelectric element 401 is controlled in accordance with the color of the cut-out target area, so that the pulsation flow having the characteristic proper to the property of the surgery target site can be ejected.

In this case, when the cut-out target area is a cut-out prohibited area, the voltage controller 32 stops the voltage signal input to the piezoelectric element 401 so as to stop the ejection of the fluid from the fluid ejection opening portion 212, so that the high-precision cut-out or incision can be performed more surely.

Furthermore, the color sensor is adopted as the color detecting unit, and thus the color of the cut-out target area can be detected with a simple construction.

Still furthermore, the piezoelectric element is adopted as the volume varying unit, and the voltage applied to the piezoelectric element is controlled in accordance with the color of the cut-out target area detected by the color sensor, so that the pressure of the fluid ejected from the fluid channel pipe can be adjusted with a simple construction.

The lookup table is referred to on the basis of the color of the cut-out target area to determine the voltage applied to the piezoelectric element, so that the characteristic of the pulsation flow to be ejected can be easily changed in accordance with the color of the surgery target area.

Furthermore, a patient beforehand takes nanocapsules which have cell selectivity of a target pathologic lesion and encapsulated with any dye. Therefore, tumor such as cancer cells or the like to be cut out can be surely colored with any color, and the property of the cut-out target area can be easily detected by determining the color of the cut-out target area through the color sensor. As comparison with a case where a general method using a catheter or direct ejection is applied as a dye marking method, the load imposed on the patient can be reduced.

### Second Embodiment

According to a second embodiment, the first embodiment is further added with a function of informing a cut-out state to the operator in accordance with the nozzle position detected by a magnetic sensor or a cut-out area detected through image processing.

### Construction

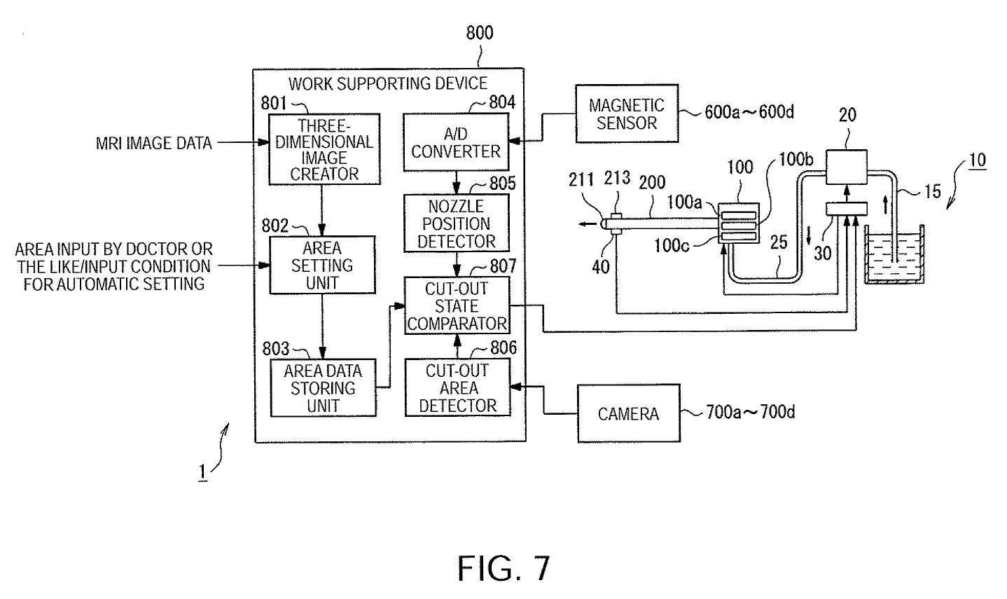

FIG. 7 is a diagram showing the construction of a water pulse surgical knife according to the second embodiment of the present invention.

The water pulse surgical knife 1 shown in FIG. 7 has the same construction as the water pulse surgical knife 1 shown in FIG. 1 except that magnetic sensors 600a to 600d, cameras 700a to 700d, a work supporting device 800 and a magnet 213 are added to the water pulse surgical knife 1 shown in FIG. 1. Accordingly, different portions in construction will be mainly described.

As shown in FIG. 7, the magnet 213 used to detect the position of the nozzle 211 is disposed in the neighborhood of the nozzle 211 of the connection fluid channel pipe 200.

The magnetic sensors 600a to 600d are magnetic sensors for measuring the three-dimensional position through magnetic detection. The magnetism of the magnet 213 disposed in the neighborhood of the nozzle 211 of the pulsation generator 100 is detected, and the information representing the detected magnetism (hereinafter referred to as “magnetic detection value”) is output to the work supporting device 800.

The magnetic sensors 600a to 600d are disposed at different positions so as to surround a patient before a surgical operation is executed on the patient. Before the surgical operation is executed, a detection reference axis for the arranged magnetic sensors 600a to 600d is set, the coordinate of the three-dimensional position measurement and the actual position of the patient are associated with each other, and the detection value is calibrated by using the magnet 213.

The cameras 700a to 700d are digital cameras which can pick up color images, and outputs pickup image data to the work supporting device 800.

The cameras 700a to 700d are arranged at different positions at which they can pick up images of an affected site when a surgical operation is carried out.

The work supporting device 800 is constructed by PC (Personal Computer) including CPU (Central Processing Unit), a memory, a hard disc, etc., and executes, in cooperation with the controller 30, the working support processing for supporting a work when a surgical operation or the like is executed by using the fluid ejection device 1.

Specifically, the work supporting device 800 has a three-dimensional image creator 801, an area setting unit 802, an area data storing unit 803, an A/D (Analog to Digital) converter 804, a nozzle position detector 805, a cut-out area detector 806 and a cut-out state comparator 807.

The three-dimensional image creator 801 creates a three-dimensional image of a surgery target site on the basis of image data representing the shape of the surgery target site (for example, brain or the like) picked up by MRI (Magnetic Resonance Imaging system) before a surgical operation is executed. The three-dimensional image creator 801 outputs the data of the created three-dimensional image to the area setting unit 802.

The area setting unit 802 sets an area to be cut out or the like in the surgical operation (cut-out permitted area), an area (cut-out prohibited area) other than the cut-out permitted area and an area (neighboring area) which is within the cut-out permitted area and within a threshold value range set from the outer edge of the cut-out permitted area.

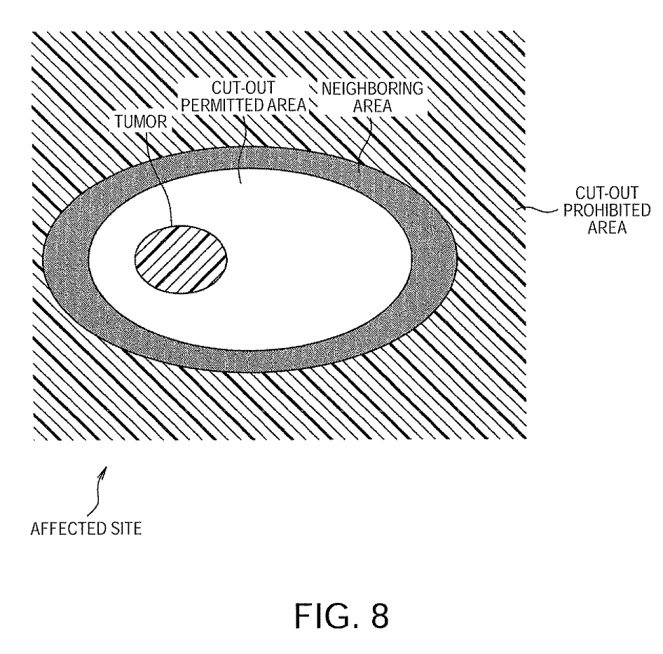

FIG. 8 is a schematic diagram showing a state that a cut-out permitted area, a cut-out prohibited area and a neighboring area are set in an affected site.

As shown in FIG. 8, an area where tumor or the like exists in an affected site is set as a cut-out permitted area, and the outside of the cut-out permitted area is set as a cut-out prohibited area in which incision or cut-out is improper. Within the cut-out permitted area, a fixed area bounding with the cut-out prohibited area is set as a neighboring area.

When these areas are set, the data of the three-dimensional image is displayed on a display unit. Therefore, a doctor can specify each area by manual input, the work supporting device 800 can automatically set each area on the basis of the color or shape of an affected site and then a doctor can acknowledge this area setting, etc.

The area setting unit 802 outputs to the area data storing unit 803 the three-dimensional image data for which the cut-out permitted area, the cut-out prohibited area and the neighboring area are set.

The area data storing unit 803 stores the data of the three-dimensional image for which the cut-out permitted area, the cut-out prohibited area and the neighboring area input from the area setting unit 802 are set.

The A/D converter 804 converts magnetic detection values input from the magnetic sensors 600a to 600d to corresponding digital values, and outputs the digital values thus converted to the noise position detector 805. The nozzle position detector 805 calculates the three-dimensional position of the magnet 213 on the basis of the respective digital values representing the magnetic detection values of the magnetic sensors 600a to 600d, thereby detecting the position of the nozzle 211.

The nozzle position detector 805 outputs a signal representing the detected position of the nozzle 211 (hereinafter referred to as “nozzle position signal”) to the cut-out state comparator 807. The cut-out area detector 806 detects an area cut out or incised through the surgical operation (hereinafter referred to as “cut-out area”) on the basis of the image data input from the cameras 700a to 700d. For example, the cut-out area detector 806 executes edge extract on the image picked up by each of the cameras 700a to 700d before the surgical operation, and detects variation of the edge in the image picked up during the surgical operation or detects variation of the color of the area surrounded by the edge, whereby the cut-out or incised area can be detected.

The cut-out area detector 806 outputs the data representing the detected cut-out or incised area (hereinafter referred to as “cut-out area data”) to the cut-out state comparator 807. The cut-out state comparator 807 sets a flag representing the state of the cut-out or incision in the surgical operation (image determining flag) on the basis of the cut-out area data input from the cut-out area detector 806 and the data of the three-dimensional image stored in the area data storing unit 803.

Furthermore, the cut-out state comparator 807 sets a flag representing the state of the nozzle position (the nozzle position determining flag) on the basis of the nozzle position signal input from the nozzle position detector 805 and the data of the three-dimensional image stored in the area data storing unit 803.

The cut-out state comparator 807 determines properness of the surgical operation on the basis of the image determining flag and the nozzle position determining flag, and outputs a signal representing the determination result to the controller 30.

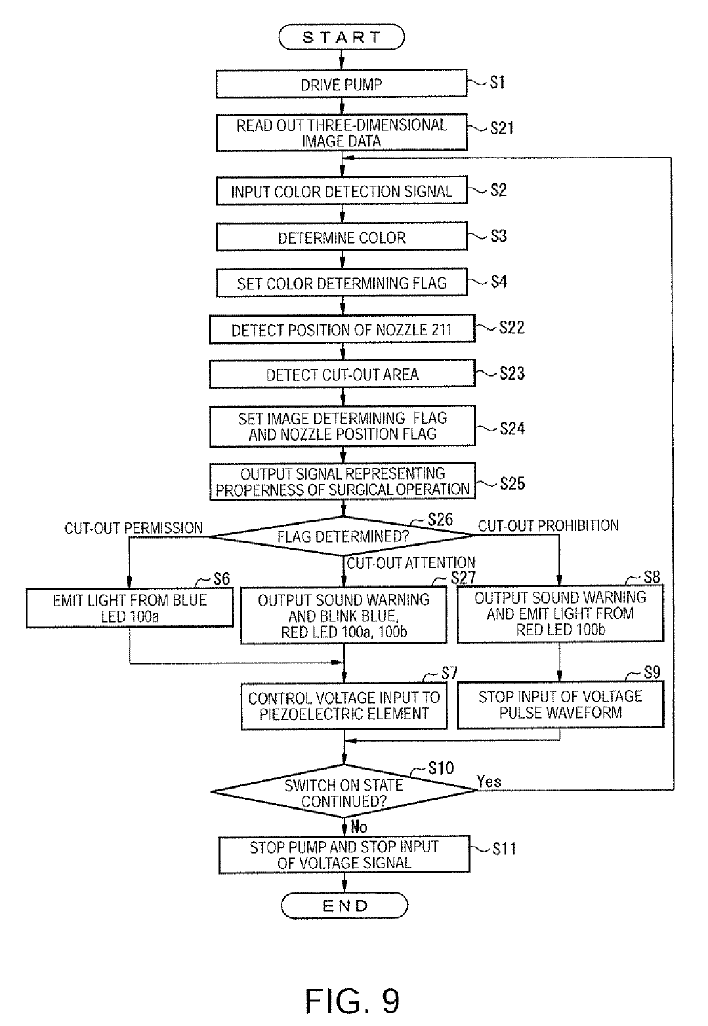

FIG. 9 is a flowchart showing the control processing executed in cooperation of the work supporting device 800 and the controller 30.

Here, the controller 30 repetitively executes the processing shown in FIG. 9 when the switch (not shown) for starting fluid ejection is set to ON state by the operator.

In the control processing shown in FIG. 9, the steps for executing the same processing as the control processing of FIG. 6 in the first embodiment are represented by the same step numbers, and different portions of the processing will be mainly described. After the processing of the step S1, the work supporting device 800 reads the three-dimensional image data stored in the area data storing unit 803 in step S21, and then shifts the processing to step S2.

Furthermore, after the processing of the step S4, the work supporting device 800 detects the position of the nozzle 211 by the nozzle position detector 805 in step S22, and then shifts to step S23 to detect a cut-out area by using the cut-out area detector 806.

Subsequently, in step S24, the work supporting device 800 determines by the cut-out state comparator 807 which one of the cut-out permitted area, the cut-out prohibited area and the neighboring area each of the cut-out area and the position of the nozzle 211 corresponds to, and sets a flag corresponding to the determination result (image determining flag and nozzle position flag).

Specifically, the cut-out state comparator 807 determines which one of the cut-out permitted area, the cut-out prohibited area and the neighboring area corresponds to the cut-out area represented by the cut-out area data in the three-dimensional image data stored in the area data storing unit 803.

When the cut-out area corresponds to a cut-out permitted area, the cut-out state comparator 807 sets a cut-out state proper flag representing that the cut-out/incision state is proper, and when the cut-out area corresponds to a cut-out prohibited area, the cut-out state comparator 807 also sets a cut-out state improper flag representing that the cut-out/incision state is improper. Furthermore, when the cut-out area corresponds to a neighboring area, the cut-out state comparator 807 sets a cut-out state attending flag representing that the cut-out/incision state is a state to which attention should be paid.

Furthermore, on the basis of the nozzle position signal input from the nozzle position detector 805 and the three-dimensional image data stored in the area data storing unit 803, the cut-out state comparator 807 determines which one of the cut-out permitted area, the cut-out prohibited area and the neighboring area corresponds to the nozzle position.

When the nozzle position corresponds to the cut-out permitted area, the cut-out state comparator 807 sets a nozzle position proper flag representing that the nozzle position is proper, and when the nozzle position corresponds to the cut-out prohibited area, the cut-out state comparator 807 sets a nozzle position improper flag representing that the nozzle position is improper. Furthermore, when the nozzle position corresponds to the neighboring area, the cut-out state comparator 807 sets a nozzle position attending flag representing that the nozzle position is under a state to which attention should be paid.

Subsequently, in step S25, the work supporting device 800 refers to the image determining flag and the nozzle position determining flag by the cut-out state comparator 807, and outputs a signal representing properness of the surgical operation to the controller 30.

Specifically, the cut-out state comparator 807 refers to the image determining flag and the nozzle position determining flag. First, when any one of the cut-out state improper flag and the nozzle position improper flag is set, the cut-out state comparator 807 outputs to the controller 30 a signal representing that the cut-out/incision state is improper (hereinafter referred to as “cut-out prohibiting signal”).

Furthermore, in a case where neither the cut-out state improper flag nor the nozzle position improper flag is set, when the cut-out state attending flag or the nozzle position attending flag is set, the cut-out state comparator 807 outputs to the controller 30 a signal representing that the cut-out/incision state is a state to which higher attention should be paid (hereinafter referred to as “cut-out attending signal”).

Furthermore, in a case where neither the cut-out state improper flag nor the nozzle position improper flag is set and also neither the cut-out state attending flag nor the nozzle position attending flag is set, when the cut-out proper flag or the nozzle position proper flag is set, the cut-out state comparator 807 outputs to the controller 30 a signal representing that the cut-out/incision state is proper (hereinafter referred to as “cut-out permitting signal”).

As described above, the cut-out state comparator 807 determines which area is a cut-out target area corresponding to an ejection target area of the pulsation generator 100, and outputs the signal corresponding to the determination result to the controller 30.

Subsequently, in step S26, the controller 30 performs the flag determination on the basis of the color determining flag set in step S4 and the signal representing the properness of the surgical operation input from the cut-out state comparator 807 in step S25.

Specifically, when the cut-out permitting flag is set as the color determining flag and the signal representing the properness of the surgical operation is the cut-out permitting signal, the processing is shifted to step S6. When the cut-out prohibiting flag is set as the color determining flag and the signal representing the properness of the surgical operation is the cut-out prohibiting signal, the processing is shifted to step S8. Furthermore, when the signal representing the properness of the surgical operation is the cut-out attending signal or the color determining flag or the signal representing the properness of the surgical operation is the cut-out prohibiting flag or the cut-out prohibiting signal, the processing is shifted to step S27.

In step S27, the controller 30 outputs a sound warning for promoting attention and blinks light emission from the blue LED 100a and the red LED 100b, and shifts the processing to the step S7.

According to the construction described above, even when the cut-out permitting flag is set on the basis of the color of the cut-out target area detected by the color sensor 40, the sound warning and the light emission are performed when the cut-out attending signal or the cut-out prohibiting signal is output on the basis of image data analysis, so that the operator is promoted to pay attention.

Furthermore, even if some defect occurs in the color sensor 40 and thus the color of the cut-out target area cannot be properly determined, the cut-out prohibiting signal or the cut-out attending signal is output on the basis of the image data analysis to output sound warning and emit light, so that the operator can be informed of the fact that the cut-out target area may correspond to a cut-out prohibited area or the neighboring area thereof.

As described above, in the water pulse surgical knife 1 according to the second embodiment, the magnet (magnet 213) disposed in the pulsation generator (nozzle 211) and the magnetic detecting unit (the magnetic sensors 600a to 600d) for detecting the magnetism of the magnet disposed in the pulsation generator are provided, the position of the pulsation generator is detected on the basis of the magnetism detected by the magnetic detecting unit, and it is determined on the basis of the detected position of the pulsation generator which one of the ejection permitted area and the ejection prohibited area corresponds to the ejection target area of the pulsation generator.

With the construction as described above, the accurate position can be simply detected by disposing the magnet in the pulsation generator.

Furthermore, the image pickup unit (cameras 700a to 700d) for picking up images of an ejection target site is provided, and it is determined on the basis of the images of the ejection target site picked up by the image pickup unit which one of the ejection permitted area and the ejection prohibited area corresponds to the ejection target area of the pulsation generator.

According to this construction, the ejection target area at which the pulsation generator aims can be easily determined by a generally used camera such as a digital camera or the like. Accordingly, the fail safe function can be added, and cut-out or incision can be performed with higher precision.

In the second embodiment, when any one of the color determining flag and the signal representing the properness of the surgical operation represents cut-out prohibition, the processing is shifted from the step S26 to the step S27 in FIG. 9, and the operator is promoted to pay attention by sound and light. However, the processing may be shifted from the step S26 to the step S8 in FIG. 9.

In this case, when at least one of the color determining flag and the signal representing the properness of the surgical operation represents cut-out prohibition, the sound warning and the light emission of the red LED 100 are performed in step S8, and also the input of the voltage pulse waveform to the piezoelectric element 401 is stopped in step S9, so that fluid can be more surely prevented from being ejected to an area other than the ejection permitted area.

Furthermore, the determining condition, the magnitude of warning, etc. can be properly set in accordance with the skill of the operator.

Furthermore, in the second embodiment, the shape detecting unit for detecting the shape of an object by irradiating the object with a laser beam is provided, and it can be determined on the basis of the shape of the ejection target site picked up by the shape detecting unit which one of the ejection permitted area and the ejection prohibited area corresponds to the ejection target area of the pulsation generator. According to the construction as described above, the ejection target area of the pulsation generator can be determined by detecting the shape of the object simply and surely.

Furthermore, a gyro sensor for detecting the position and attitude of the pulsation generator may be provided to the pulsation generator. In this case, on the basis of the position and attitude of the pulsation generator which are detected by the gyro sensor, it can be determined which one of the ejection permitted area and the ejection prohibited area corresponds to the ejection target area of the pulsation generator. According to this construction, the pulsation generator itself can be provided with the function of detecting the position and attitude thereof, and thus the construction of the device can be simplified.

In the respective embodiments described above, the color sensor is applied as the color detecting unit. However, a camera which can pick up a color image may be applied as the color detecting unit. Furthermore, in the respective embodiments described above, it can be determined in accordance with the color of the cut-out target area detected by the color sensor whether the cut-out target area is a cut-out permitted area or a cut-out prohibited area. This determination may be performed by using image data picked up by a camera in addition to the color detected by the color sensor. Accordingly, the control which is more suitable for the property of the cut-out target area can be performed.

In the above embodiments, one pulsation generator 100 for ejecting fluid is provided. However, a plurality of pulsation generator 100 may be provided.

Furthermore, in the above embodiments, this invention is applied to a water pulse surgical knife for surgical operation. However, as described above, this invention may be applied to cleaning of minute objects and structures, cutting or cut-out of objects, etc. For example, in a case where this invention is applied to a cleaning device for cleaning stain or rust adhering to an object, when a color other than a background color (the original color of an object) is detected, water, cleaning agent or the like can be ejected with a characteristic which is varied in accordance with the type of stain, and thus the working efficiency can be enhanced.

What is claimed is:

1. A fluid ejection device comprising:
a fluid chamber into which fluid flows;
a volume varying unit that varies the volume of the fluid chamber;
an inlet fluid channel and an outlet fluid channel intercommunicating with the fluid chamber;
a fluid ejection port that ejects fluid flowing out from the outlet fluid channel;
a fluid supply unit that supplies fluid into the inlet fluid channel;
a controller that controls the variation of the volume of the fluid chamber by the volume varying unit; and
a color detector that detects color of a fluid ejection target area facing the fluid ejection port, wherein the controller controls the variation of the volume of the fluid chamber by the volume varying unit in accordance with the color detected by the color detecting unit.

2. The fluid ejection device according to claim 1, wherein the controller controls the variation of the volume of the fluid chamber by the volume varying unit so that discharge of fluid generated by the variation of the volume of the fluid chamber is stopped when the color detected by the color detecting unit is a color representing an ejection prohibited area at an ejection target site.

3. The fluid ejection device according to claim 1, wherein the controller controls the variation of the volume of the fluid chamber by the volume varying unit so that a pulsation flow having a characteristic changeable in accordance with the color detected by the color detecting unit is ejected when the color detected by the color detecting unit is a color representing an ejection permitted area at an ejection target site.

4. The fluid ejection device according to claim 1, further comprising:
a shape data obtaining unit that obtains data representing the shape of a fluid ejection target site;
an area setting unit that sets an ejection permitted area and an ejection prohibited area at an ejection target site on the basis of the data representing the shape of the ejection target site obtained by the shape data obtaining unit;
an area determining unit that determines which one of the ejection permitted area and the ejection prohibited area corresponds to the fluid ejection target area; and
a discharge suppression unit that suppresses discharge of fluid generated by the variation of the volume of the fluid chamber when the area determining unit determines that the fluid ejection target area corresponds to the ejection prohibited area.

5. The fluid ejection device according to claim 1, further comprising:
an informing unit that informs to an operator (user) by using at least one of sound and light, wherein the informing unit informs to the operator when the fluid ejection target area corresponds to any one of an ejection prohibited area and a neighboring area corresponding to an area in the neighborhood of the ejection prohibited area.

6. A fluid ejection method comprising:
supplying fluid into a fluid chamber; ejecting pressurized fluid from a fluid ejection port by varying the volume of the fluid chamber;
detecting color of a fluid ejection target area facing the fluid ejection port; and
controlling the variation of the volume of the fluid chamber in accordance with the detected color.

7. A fluid ejection surgical instrument comprising:
a fluid chamber into which fluid flows;
a volume varying unit for varying the volume of the fluid chamber;
an inlet fluid channel and an outlet fluid channel intercommunicating with the fluid chamber;
a fluid ejection port for ejecting fluid flowing out from the outlet fluid channel;
a fluid supply unit for supplying fluid into the inlet fluid channel;
a controller for controlling the variation of the volume of the fluid chamber by the volume varying unit; and
a color detector for detecting color of a surgery target site facing the fluid ejection port, wherein the controller controls the variation of the volume of the fluid chamber by the volume varying unit in accordance with the color detected by the color detecting unit, and incises or cuts out the surgery target site with the fluid which is ejected from the fluid ejection port in accordance with the variation of the volume of the fluid chamber by the volume varying unit.
